(19) United States
(12) Patent Application Publication
(10) Pub. No.: US 2018/0214619 A1
(43) Pub. Date: Aug. 2, 2018

(54) Flexible Protection Device For Circulatory Support Device And Related Systems And Methods

(71) Applicant: Scivad LCC, Eden Prairie, MN (US)

(72) Inventor: Martin Cook, Eden Prairie, MN (US)

(21) Appl. No.: 15/882,205

(22) Filed: Jan. 29, 2018

Related U.S. Application Data
(60) Provisional application No. 62/451,955, filed on Jan. 30, 2017.
(60) Provisional application No. 62/583,594, filed on Nov. 9, 2017.

(51) Int. Cl. A61M 1/10; A61M 1/12

(52) U.S. Cl. CPC A61M 1/1008; A61M 1/122

(57) ABSTRACT

A protection device that couples to a cannula of a circulatory support device and can be used to isolate the interface of the cannula with an incision through which the cannula is disposed, and related systems and methods.

### CROSS-REFERENCE TO RELATED APPLICATION(S)

This application claims the benefit under 35 U.S.C. § 119(e) to U.S. Provisional Application 62/451,955, filed Jan. 30, 2017 and entitled “Flexible Skirt for Circulatory Support Devices and Related Systems and Methods,” and U.S. Provisional Application 62/583,594, filed Nov. 9, 2017 and entitled “Flexible Skirt for Circulatory Support Devices and Related Systems and Methods,” both of which are hereby incorporated herein by reference in their entireties.

### FIELD OF THE INVENTION

The present invention relates to mechanical circulatory support for weakened and/or ailing hearts and more specifically to circulatory support devices and methods, devices, and/or components for improving the interface between such devices and a patient's heart.

### BACKGROUND OF THE INVENTION

Mechanical circulatory support has become a standard of practice for the treatment of late-stage heart failure. The most common method of providing mechanical circulatory support is a left ventricular assist device (“LVAD”), which is a pump that takes over much of, if not all, the function of the left ventricle. LVADs may use various mechanisms of action but typically involve a rotary pump placed outside the heart, either in the thoracic cavity or in a sub-diaphragmatic pocket. The placement of the pump outside the heart requires that blood be drawn from the heart. As shown in FIG. 1, to achieve this, the LVAD 10 typically has a cannula 12 that is inserted into the ventricle 14 through the heart wall and remains in place for the duration of therapy, often for many years. It is understood that the cannula 12 can be an integral component of the LVAD 10 or, alternatively, can be a separate, detachable component. That is, it is understood that other known circulatory support (or “heart assist”) devices have a cannula, tube, or any other type of conduit that is integral with the device and is positioned through the heart wall and into the ventricle 14. Further, other known devices have a pump (or impeller) that is disposed within that conduit. Regardless of the type of circulatory support device, it is understood that each has a conduit that is disposed through the organ wall.

As an alternative to the LVAD, there are circulatory support devices that support the right ventricle, known as right ventricular assist devices (“RVADs”) and devices that support both ventricles simultaneously (“BiVADs”). These devices may draw blood from the atria rather than the ventricles. Regardless, in each case, if the device is positioned outside the heart, then a conduit is typically provided for blood to flow from the heart into the pump, and hence is typically referred to as an inflow conduit.

It is understood that the terms “conduit” and “cannula” are used interchangeably herein. Both terms are intended to mean any structure that is positioned in a chamber through an organ wall (such as a ventricle through a heart wall) and allows for intake of blood, regardless of the type of circulatory support device of which the structure is a component.

These inflow conduits of any known form suffer many drawbacks that may result in complications of therapy and adverse events that deleteriously affect the patients undergoing treatment. One such drawback is the risk of generating small particles that can result in emboli. Placement of an inflow conduit requires that a large hole be made in the heart wall, typically at or near the apex of the left ventricle as shown in FIG. 1. Typically, this involves coring a cylindrical hole in the wall of the ventricle using a circular cutting tool, inserting the conduit into the hole, and affixing or otherwise attaching the conduit to the heart wall. The cutting of the heart wall, including, for example, the myocardium and/or other layers of the wall, may release small particles—either at the time of the procedure or shortly afterwards - that may result in emboli and contribute to ischemic stroke complications, which are known to be a significant problem for patients undergoing circulatory support.

Another drawback relates to the development of thrombosis that forms around the conduit and the chamber inner wall (endocardium) adjacent to and/or in contact with the conduit where the conduit is positioned through the heart wall and protrudes into the heart chamber. It is known that the damage to the endocardium as well as changes in blood flow within the heart chamber lead to the formation of thrombosis and in particular thrombosis that adheres to the wall of the heart, which is called mural thrombosis. It is clear that the insertion and placement of a conduit for circulatory support involves significant trauma to the endocardium as well as altered blood flow. In particular, blood flow in the chamber may be stagnated around the base of the conduit where it extends out of the heart wall into the chamber. A common complication is the formation of a “wedge thrombus,” which is a thrombus that forms around the conduit at the interface of the cannula and the heart wall. These wedge thromboses at the interface of the inflow conduit and the heart wall—including, for example, the myocardium layer thereof—are considered to be one of the significant sources of thromboembolic complication. Devices have been developed with textured surfaces that aim to promote tissue ingrowth of the cylindrical wound through the heart wall and also to promote the development of neointima along the outer blood-contacting surfaces of the device. It is believed that these textured surface modifications may prevent thrombus formation and promote neointimal tissue with endothelialization and angiogenesis. Also, the textured surfaces may promote attachment to the conduit of any thrombosis that does form, with the hope that it remains adhered, thus preventing embolization. Despite these measures, embolization of thromboses remains a significant issue and risk for these circulatory support devices.

It is known that damage to the endothelium serves as a nidus for platelet aggregation and fibrin formation and may be related to observed fibrin deposits on the blades of rotary blood pumps and other thromboembolic complications. The damage to the endocardium exposes the blood to the underlying collagen in the heart wall and this can promote clot formation. Further, movement of an LVAD pump relative to the heart as a result of patient movement as well as changes in size of the heart, as a result of therapy, may result in renewed damage to the epicardium over time and the aforementioned sequalae.

There is a need in the art for an improved device or component for addressing the above complications of LVADs and other similar heart assist devices.

### BRIEF SUMMARY OF THE INVENTION

Discussed herein are various protection devices, components, and methods for use with heart assist devices.

In Example 1, a protection device for isolating a cannula/incision interface from organ chamber fluid comprises a flexible sheet, and an inner opening defined by the sheet, wherein the inner opening is sized to receive a cannula such that contact is established between the sheet and the cannula when the cannula is disposed through the inner opening. A portion of the sheet is contactable with an inner wall of an organ chamber.

Example 2 relates to the protection device according to Example 1, wherein the contact between the sheet and the cannula is snug contact.

Example 3 relates to the protection device according to Example 1, wherein the portion of the sheet is an outer edge of the sheet, wherein the outer edge is snuggably contactable with the inner surface of the organ chamber.

Example 4 relates to the protection device according to Example 1, further comprising at least two ribs operably coupled to the flexible sheet.

Example 5 relates to the protection device according to Example 4, wherein each of the at least two ribs extends from the inner opening to the outer edge of the flexible sheet.

Example 6 relates to the protection device according to Example 5, wherein the at least two ribs are constructed and arranged to move between a retracted position in which the at least two ribs and the flexible sheet are disposed adjacent to the cannula and a deployed position in which the outer edge is in contact with the inner wall of the organ chamber.

Example 7 relates to the protection device according to Example 1, further comprising a stem operably coupled to the flexible sheet, wherein the stem defines an opening sized to receive the cannula.

Example 8 relates to the protection device according to Example 7, wherein the stem is operably coupled to the flexible sheet at or near the inner opening.

Example 9 relates to the protection device according to Example 7, wherein the stem comprises an anchor disposed at or near a proximal end of the stem.

Example 10 relates to the protection device according to Example 9, wherein the anchor comprises a lip.

In Example 11, a protection device for use with a cannula disposed through a wall of an organ comprises a flexible sheet comprising a retracted configuration and a deployed configuration, a cannula opening defined in the sheet, the cannula opening sized to receive the cannula, and a contactable portion of the circular sheet, the contactable portion being shaped to be contactable with an inner wall of the organ when the sheet is in the deployed configuration.

Example 12 relates to the protection device according to Example 11, wherein the contactable portion snugly contacts the inner wall of the organ when the sheet is in the deployed configuration.

Example 13 relates to the protection device according to Example 11, wherein the contactable portion forms a substantially fluidic seal with the inner wall of the organ when the sheet is in the deployed configuration.

Example 14 relates to the protection device according to Example 11, wherein the deployed configuration comprises a substantially conical or rounded shape.

Example 15 relates to the protection device according to Example 11, wherein the deployed configuration comprises a substantially flat shape.

Example 16 relates to the protection device according to Example 11, wherein the cannula opening is sized to receive the cannula in a snug coupling.

In Example 17, a protection device for use with a cannula configured for insertion through a wall of an organ of a patient comprises a flexible, substantially circular sheet, a cannula opening defined in the sheet, the cannula opening sized to receive the cannula in a snug coupling, an outer edge of the circular sheet, and at least two ribs operably coupled to the circular sheet, wherein each of the at least two ribs extends from an edge of the cannula opening to the outer edge of the circular sheet, wherein the at least two ribs are constructed and arranged to move between a retracted position in which the at least two ribs and the circular sheet are disposed adjacent to the cannula and a deployed position in which the outer edge is in contact with an inner wall of the organ.

Example 18 relates to the protection device according to Example 17, wherein the outer edge is in snuggable contact with the inner wall of the organ in the deployed position.

Example 19 relates to the protection device according to Example 17, further comprising a stem operably coupled to the flexible sheet at or near the cannula opening, wherein the stem defines an opening sized to receive the cannula.

Example 20 relates to the protection device according to Example 19, wherein the stem comprises an anchor disposed at or near a proximal end of the stem.

While multiple embodiments are disclosed, still other embodiments of the present invention will become apparent to those skilled in the art from the following detailed description, which shows and describes illustrative embodiments of the invention. As will be realized, the invention is capable of modifications in various obvious aspects, all without departing from the spirit and scope of the present invention. Accordingly, the drawings and detailed description are to be regarded as illustrative in nature and not restrictive.

### BRIEF DESCRIPTION OF THE DRAWINGS

FIG. 1 is a cross-sectional schematic view of a prior art LVAD device positioned in a heart wall of a patient.

FIG. 2 is a cross-sectional schematic view of a protection device coupled to a heart assist device, according to one embodiment.

FIG. 3A is a top view of a protection device, according to one embodiment.

FIG. 3B is a side view of the protection device of FIG. 3A, according to one embodiment.

FIG. 4A is a cross-sectional schematic view of a protection device coupled to a heart assist device, according to another embodiment.

FIG. 4B is a cross-sectional schematic view of another protection device coupled to a heart assist device, according to a further embodiment.

FIG. 5 is a cross-sectional schematic view of a further protection device coupled to a heart assist device, according to yet another embodiment.

FIG. 6A is a perspective view of a protection device, according to one embodiment.

FIG. 6B is a cross-sectional schematic view of the protection device of FIG. 6A coupled to a heart assist device, according to one embodiment.

FIG. 7A is a cross-sectional schematic view of a protection device in an undeployed configuration coupled to a heart assist device, according to one embodiment.

FIG. 7B is a cross-sectional schematic view of the protection device of FIG. 7A in a deployed configuration coupled to a heart assist device positioned in a heart wall of a patient, according to one embodiment.

FIG. 7C is another cross-sectional schematic view of the protection device of FIG. 7A in a deployed configuration coupled to a heart assist device positioned in a heart wall of a patient, according to one embodiment.

FIG. 8A is a cross-sectional schematic view of a protection device in an undeployed configuration coupled to a heart assist device, according to one embodiment.

FIG. 8B is a cross-sectional schematic view of the protection device of FIG. 8A in a deployed configuration coupled to a heart assist device positioned in a heart wall of a patient, according to one embodiment.

FIG. 8C is a cross-sectional schematic view of the protection device of FIG. 8A in a restrained position via a restraint device coupled to a heart assist device, according to one embodiment.

### DETAILED DESCRIPTION

The various embodiments disclosed or contemplated herein relate to an protection or separation device that is used with a conduit for covering the interface between the conduit and the opening in the organ wall through which the conduit is positioned. For example, according to some implementations, the conduit is a component of a circulatory support device, the organ is the heart, and the chamber is the left ventricle. In such embodiments, the protection device blocks or separates the interface of the conduit and the incision from the blood in the ventricle, thereby helping to prevent particulates from being released from the incision and becoming an embolism within the blood flow. That is, the protection device effectively isolates the incision where the conduit is positioned into the chamber of the heart from the blood within the chamber and mitigates the known risks related to platelet activation. Further, the device in certain implementations can be substantially conically shaped or curvedly shaped such as to provide a rounded wall between the conduit and the wall of the heart in such a way that stagnation of the blood flow around the conduit extending from the incision is mitigated. Alternatively, the device can be substantially flat or take on any other configuration or shape while fluidically isolating the interface.

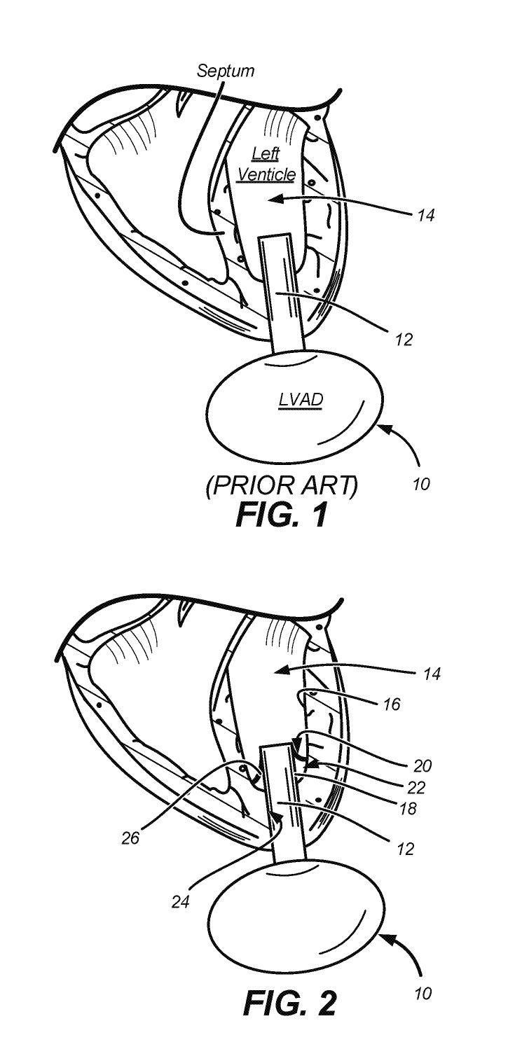

As shown in FIG. 2, in certain implementations, the protection device (also referred to as a “separation device,” “skirt device,” “skirt,” or “flexible skirt device”) 20 has a flexible sheet (also referred to as a “shield” or “membrane”) 26 that is disposed around outer surface 18 of the cannula 12 of the heart assist device 10, which in this case is an LVAD 10. It is understood that the use of the term “skirt” is intended to broadly refer to any protection device embodiment as described herein that can be used to separate the incision where the conduit is positioned into the chamber of the heart from the blood within the chamber. The flexible sheet 26 extends from the cannula 12 to the inner wall 16 of the ventricle 14, thereby separating the cannula 12 and the opening 24 from the blood in the ventricle 14. It is understood that the opening 24 (or any other organ opening through which a cannula can be positioned) is an incision, wound, or other type of opening formed by a medical professional, including, for example, a physician or surgeon, to position the cannula through the wall of the organ and into a chamber therein. In certain implementations, the deployment or positioning of the device 20 to form the isolation or enclosure 22 results in the sheet 26 forming a substantially curved or conical shape as depicted in FIG. 2. Alternatively, the sheet 26 can form a fairly flat configuration while in use. For example, in one specific implementation, the sheet 26 can be fenestrated such that the sheet 26 conforms to the shape of the inner wall 16 of the ventricle 14 while still maintaining a substantial isolation of the cannula 12/incision 24 interface from the fluids, including blood, in the ventricle 14. The flexibility of the sheet 26 allows the sheet 26 to take on any shape necessary to form the separation or enclosure 22 and thereby prevent fluidic access to the interface of the cannula 12 and the incision 24.

It is understood that the term “sheet,” for purposes of this application and the various embodiments herein, is intended to mean any sheet, disk, membrane, or laminate of flexible material that can be coupled to or positioned over a cannula according to the various implementations disclosed or contemplated herein and thereby cover the interface between the cannula and the wall opening. As discussed above, the skirt can take on a substantially conical or curved shape, a substantially or somewhat flat shape, or any other shape or configuration, including the other shapes or configurations according to the other embodiments herein, while separating the interface from the fluid within the organ. In certain embodiments, the sheet in the various embodiments disclosed or contemplated herein (including, for example, sheet 26 above) is made of Dacron, polyester, silicone, ePTFE, or any other flexible polymeric material. Alternatively, the sheet can be made of any other known biocompatible, hemocompatible, substantially impermeable, and flexible material.

According to one embodiment, the conical or curved shape of the skirt device 20 or any other protection device embodiment disclosed or contemplated elsewhere herein creates a smooth, curve-like shape around the cannula 12 as shown, thereby resulting in greater blood flow at that location around the cannula 12 in contrast to a procedure in which a skirt is not used. That is, when a cannula is inserted into the left ventricle (such as, for example, as shown in FIG. 1), the sharp angles (up to and including 90 degree angles) created by the cannula disposed within the ventricle can stagnate blood flow in that area. In contrast, the skirt device 20 as shown in FIG. 2 eliminates those sharp angles, creating a smooth feature where the cannula 12 extends into the chamber 14 and thereby reduces or eliminates the stagnation of the blood flow. Further, the sheet 26 is in contact with both the cannula 12 and the inner wall 16 of the ventricle 14 so as to isolate the incision 24 with the cannula 12 disposed therein from the blood in the ventricle 14. In other words, the incision 24 is not in fluid communication with the blood inside the ventricle 14. In certain embodiments, the contact of the sheet 26 with the cannula 12 and inner wall 16 does not establish a fluidic seal, but provides sufficient isolation to prevent any particulates from the incision 24 entering the blood in the ventricle 14. Alternatively, the sheet 26 can establish a fluidic seal or near-fluidic seal.

It is understood that the separation or isolation of the cannula/wall interface from the blood in certain implementations of the various devices disclosed or contemplated herein, including the device 20 discussed above, is not a fluidic seal. That is, the contact between the sheet 26 and the cannula 12 and separately between the sheet 26 and the inner wall 16 is sufficient to prevent most blood from entering the enclosure 22, and, more importantly, to prevent particulates from entering the blood in the ventricle 14. However, this separation or isolation is not configured to prevent all fluid—such as blood—from passing from the chamber 14 into the enclosure 22. That is, the contact between the sheet 26 and the cannula 12 and the contact between the sheet 26 and the inner wall 16 do not necessarily establish fluidic seals that prevent passage of all fluid. Instead, the isolation is sufficient to prevent most fluid from accessing the enclosure 22, such that the device 20 substantially isolates the enclosure 22 from the chamber 14, because there is sufficient contact between the cannula 12 and the sheet 26 and separately between the sheet 26 and the inner wall 16. As such, the contact sufficient to prevent passage of most but not all fluid can be referred to herein as “snug” contact (rather than fluidically sealed contact). Similarly, it is understood that the sheet 26, or any other sheet or device embodiment herein, can have one or more small openings defined or otherwise formed therein—such as the fenestrated sheet discussed above—that allow some fluid to pass through the sheet 26 while still maintaining the necessary physical protection against passage of particulates into the ventricle 14. That is, despite various skirt embodiments allowing some fluid to pass from the chamber 14 into the enclosure 22, it is understood that the isolation in the various embodiments herein is sufficient to prevent the passage of most or all particulates from the enclosure 22 into the chamber 14, thereby preventing or reducing the risk of an embolism in the blood flow.

In accordance with certain embodiments with respect to any of the device implementations disclosed or contemplated herein, the device (such as device 20 or any other protection device herein) does not provide structural support to the cannula 12. That is, the flexibility of the sheet 26 is not configured to provide any type of structural support for the cannula 12 in relation to the inner wall 16 or any other portion of the chamber 14.

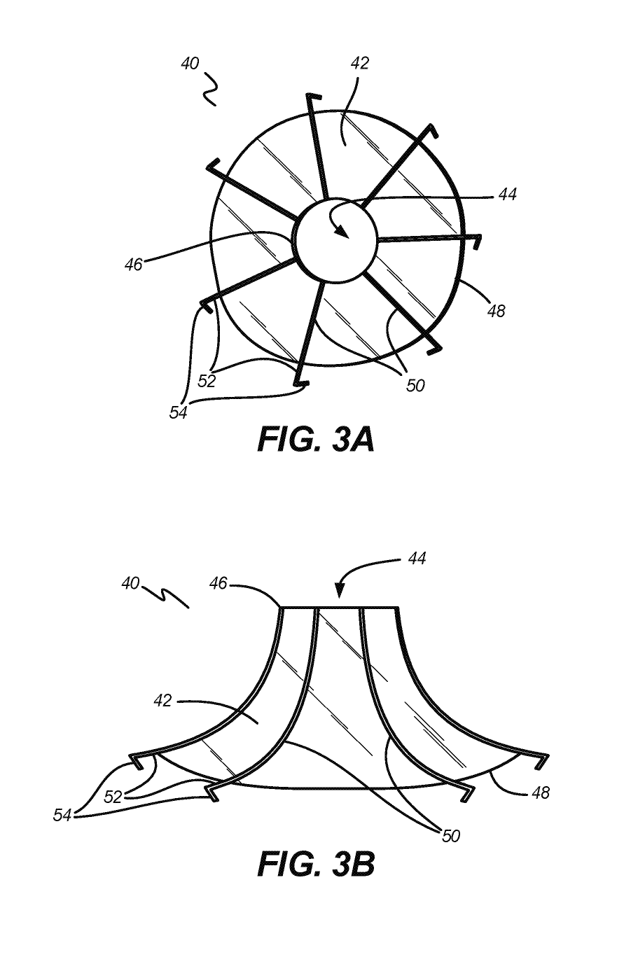

FIGS. 3A and 3B provide a top view (FIG. 3A) and a side view (FIG. 3B) of a skirt device 40 according to another embodiment. In this implementation, the device 40 has a sheet 42 of flexible material formed in a substantially conical shape. The device 40 also has a cannula opening 44 defined within the sheet 42 in the center of the cone-shaped sheet 42 as shown. It is understood that the inner edge 46 of the sheet 42 (which defines the opening 44) is sized to be positioned around a cannula (not shown) (such as cannula 12) (or have the cannula positioned therethrough) such that the outer surface of the cannula and the inner edge 46 of the sheet 42 are in contact. Further, the sheet 42 has an outer edge 48 that contacts the inner wall of the chamber when the device 40 is positioned within the organ chamber (such as, for example, the left ventricle 14 discussed above) and contacts the inner wall to create separation of the cannula/wall interface from the chamber fluids, as discussed in further detail above with respect to skirt device 20. As such, the contact established between the inner edge 46 and the cannula (not shown) and the contact established between the outer edge 48 and the inner wall of the chamber (not shown) result in substantial isolation of the interface between the cannula (not shown) and the wall opening (not shown) such that the skirt device 40 prevents any particulates from that interface gaining access to the organ chamber.

According to certain alternative embodiments, the inner edge 46 can have a suture, an elastic material, or a shape-memory material coupled thereto (or the edge 46 can be made of such materials) such that the inner edge 46 is tensioned or urged—or can be urged by a user—into a smaller circumference when the cannula (not shown) is positioned therethrough, thereby ensuring a good fit between the cannula (not shown) and the cannula opening 44 and resulting in stronger contact between the edge 46 and the cannula (not shown) and thus a stronger isolation and, in some implementations a near-fluidic or fluidic seal therebetween.

According to an alternative implementation, the protection device 40 can also have ribs 50 extending radially from the inner edge 46 to the outer edge 48 of the device 40. The ribs 50 can provide structure and/or support to the sheet 42, thereby helping to form the conical or curved shape as discussed above. Further, the ribs 50, in certain embodiments, can help the device 40 move between the deployed configuration (as shown in FIGS. 3A and 3B, for example), and an undeployed configuration in which the outer edge 48 of the sheet 42 is positioned adjacent to or in contact with the cannula (such as cannula 12). That is, the ribs 50 can be made of a shape-memory material such that the ribs 50 are configured to be in an untensioned state when the ribs 50 are positioned in the deployed configuration (the conical shape) as shown in FIGS. 3A and 3B. As such, force is required to move the ribs 50 into and/or maintain the ribs 50 in the undeployed state in which the outer edge 48 is disposed against or adjacent to the outer surface of the cannula as discussed above. Alternatively, the ribs 50 are not made of a shape-memory material but are moveable between deployed and undeployed configurations.

In one embodiment, there are seven ribs 50 coupled to the sheet 42. Alternatively, there can be any number of ribs 50 ranging from two or three ribs up to any number of ribs 50 that can be coupled to the sheet 42.

According to a further alternative, each of the ribs 50 can extend radially beyond the outer edge 48 of the sheet 42 as shown in FIGS. 3A and 3B. In such implementations, the distal ends 52 of the ribs 50 can be used to attach or anchor the skirt device 40, and more specifically, the outer edge 48 to the inner wall of the organ chamber (such as the inner wall 16 of the left ventricle 14 as discussed above). That is, the distal ends 52 of the ribs 50 can serve as attachment devices that are inserted into the tissue of the inner wall 16, thereby attaching or anchoring the skirt device 40 to the inner wall 16 and thus establishing or strengthening the contact between the outer edge 48 and the inner wall 16 and thus the isolation created by the sheet 42.

In yet another alternative implementation, the ribs 50 can also have barbs 54 or other known attachment devices or mechanisms extending from the distal end 52 of each rib 50. In one embodiment, the barbs 54 can be used to attach or anchor outer edge 48 to the inner wall by inserting the barbs 54 into the tissue of the inner wall, thereby establishing or further strengthening the contact between the outer edge 48 and inner wall 16.

It is understood that any of the protection device embodiments disclosed or contemplated herein can have ribs substantially similar to the ribs 50 or any variations thereof described above. Further, in those embodiments having ribs (such as those discussed above), the ribs can be made of a shape-memory material such as nitinol. Alternatively, the ribs can be made of any other known deployable material in order to ensure the flexible sheet/skirt structure takes on a smooth shape and provides sufficient contact—up to and including a fluidic seal in some implementations—with both the endocardium and the cannula.

In certain alternative embodiments, the skirt device (such as skirt device 40) can have a different shape other than substantially circular as shown in FIG. 3A. That is, the skirt device can have other shapes such as an elliptical shape, which may be more suitable to match the shape of the chamber (such as the ventricular shape) and/or the shape of the cannula (not shown) around which the skirt device is designed to fit.

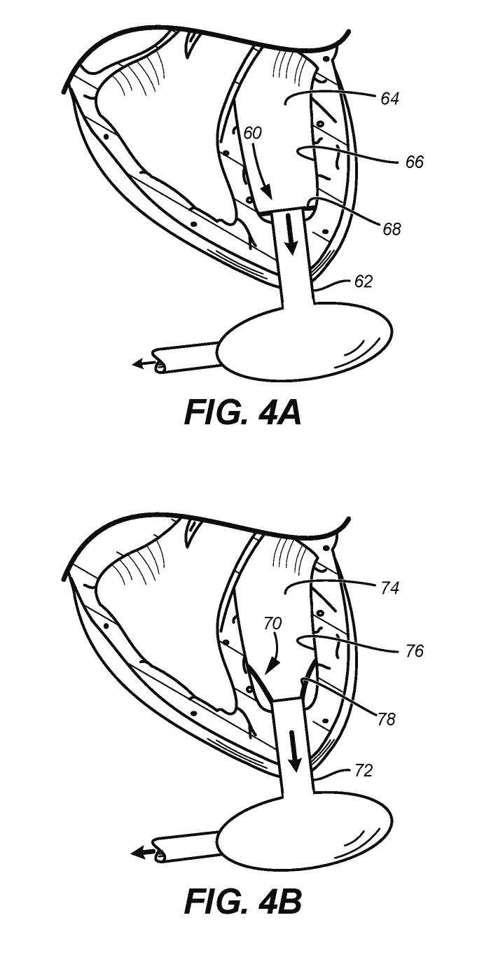

FIGS. 4A and 4B depict two additional implementations in which an protection device is attached to the cannula at the distal end thereof. For example, the skirt device 60 in FIG. 4A has a flexible sheet 68 that is attached to and extends from the distal end of the cannula 62, thereby allowing for the cannula 62 to be positioned flush, or substantially flush, with the inner wall 66 of the ventricle 64 and thus not extend into the heart chamber 64. Alternatively, the cannula 62 with the skirt device 60 attached at the distal end thereof can be positioned such that the distal end extends only a short distance into the chamber 62 as shown. In such embodiments, the skirt device 60 has a generally flat configuration as shown.

FIG. 4B depicts a skirt device 70 having a flexible sheet 78 that is attached to and extends from the distal end of the cannula 72 and is attached to the inner wall 76 of the ventricle 74, according to another embodiment. In this implementation, the flexible sheet 78 is attached to the inner wall 76 such that the flexible sheet 78 extends from the cannula 72 in a funnel-shaped configuration directed toward the cannula 72 as shown. In accordance with certain embodiments, this funnel-shaped configuration reduces or prevents thrombus formation by eliminating any areas, edges, or corners in which blood flow can be confined to a small space such that shear stresses may become very low.

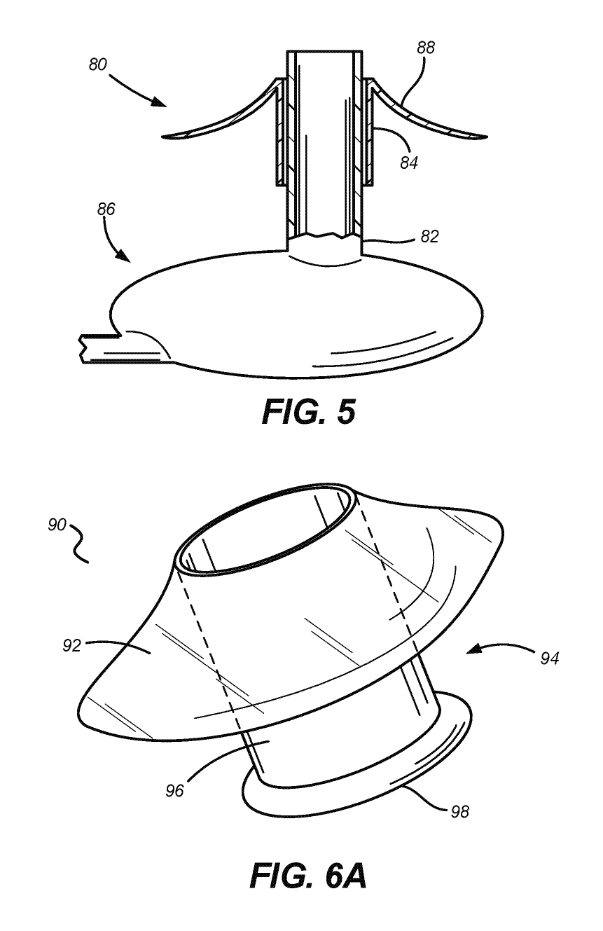

As depicted in FIG. 5, certain protection device 80 implementations attach to the cannula 82 not at an edge of the device 80 as described with other implementations above but along a length 84 of the device 80. That is, the skirt device 80 can have an interior length (also referred to herein as a “stem”) 84 that is positioned against and attached to a portion of the outer surface of the cannula 82 and the flexible sheet 88 can be coupled to the stem 84 as shown. According to one embodiment, the entire device 80 can be made of flexible material. That is, both the stem 84 and the sheet 88 can be made of a single, integral flexible material. Alternatively, the sheet 88 can be made of flexible material and can be attached to the stem 84, which can be made of a different material. In one embodiment, the stem 84 is attached to the cannula 82 via bonding, adhesive, or some other known mechanical fixation means. When attached, the stem 84, according to one embodiment, provides positioning support for the skirt device 80 by maintaining the position of the skirt device 80 in relation to the cannula 82. Because the stem 84 is permanently fixated to the cannula 82 in certain implementations, the skirt device 80 is removed when the assist device 86 is removed for repair or replacement (or any other reason). In one embodiment, the cannula 82 can be a separate component that is removable from or attachable to the assist device 86, thereby allowing for replacement of the cannula 82 with the skirt device 80 attached with another cannula and skirt device (not shown).

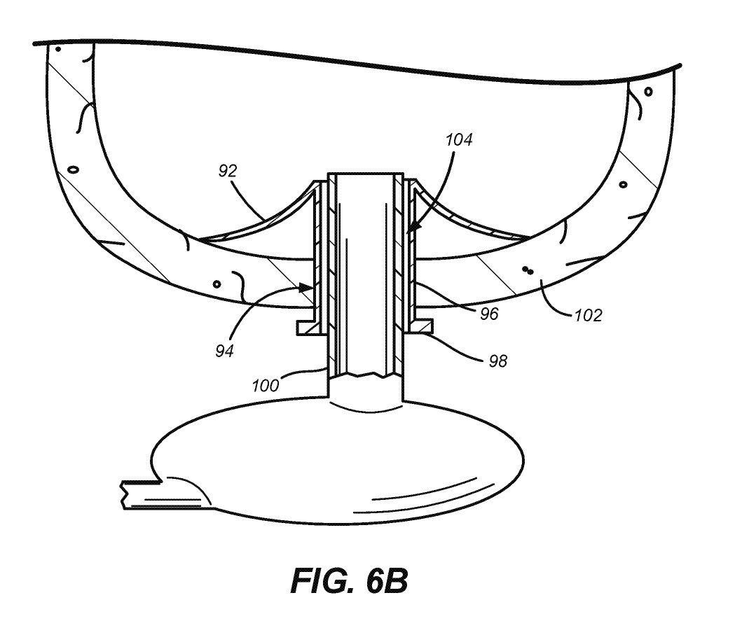

FIGS. 6A and 6B depicts an alternative embodiment of an protection device 90 in which the device 90 is not permanently attached to or integral with the cannula 100 (as shown in FIG. 6B). In this embodiment, the device 90 has an anchor 94 to prevent the skirt device 90 from moving in relation to the opening 104, which in this example is an incision 104. More specifically, the skirt device 90 has a sheet (also referred to herein as a “skirt structure”) 92 coupled to an anchor 94 that has a stem 96 and an attachment structure 98. According to one embodiment, this specific skirt device 90 configuration with the skirt structure 92 and anchor 94 has a grommet-like shape. In use, as best shown in FIG. 6B, the skirt device 90 is typically positioned around the cannula 100 such that the skirt structure 92 is disposed at or near the distal end of the cannula 100 (or some distance proximally therefrom) while the stem 96 is disposed through the opening 104 in the organ wall 102 and the attachment structure 98 is disposed outside the wall 102.

According to one implementation, the attachment structure 98 is a rim or lip 98 that has a thickness that is larger than the gap between the cannula 100 and the opening 104, thereby preventing the rim 98 from passing through that gap and thereby helping to anchor the device 90 in place. Alternatively, the attachment structure 98 can be one or more hooks, barbs, prongs, or any other known anchoring, fixation, or retention device that can anchor the device 90 in place in relation to the organ wall 102.

The stem 96 in one embodiment can assist with the healing of the wound produced by forming the opening 104 while helping to isolate or separate the wound and limit preoperative bleeding. That is, the stem 96 in this specific example can have a textured layer (as discussed in additional detail elsewhere herein) on its outer surface facing the opening 104 such that the layer acts like a known wound pad to absorb bleeding, apply compression, and promote rapid healing of the wound formed through the heart wall.

In certain embodiments, the entire skirt device 90, including the skirt structure 92, the stem 96, and the attachment structure 98, is flexible and compressible such that the device 90 can be deformed or compressed and thereby inserted into and positioned in the wall opening 104. Thus, in use, according to one embodiment, the device 90 can first be positioned as shown in FIG. 6B within the opening 104 after formation of that opening 104 and prior to positioning of the cannula 100 therein. Once the device 90 is positioned as desired, the cannula 100 can then be inserted through the opening defined in the device 90 by the stem 96 such that the cannula 100 is positioned as shown in FIG. 6B. It is understood that the skirt structure 92 and the stem 96 are configured to establish contact with the outer surface of the cannula 100, thereby separation or isolating the opening 104 from the blood in the chamber 106 and other bodily fluids as discussed in further detail above.

According to further embodiments, this specific device 90 also allows for removal and/or replacement of the cannula 100 without having to remove the skirt device 90. That is, in the same fashion that the cannula 100 can be positioned through the organ wall 102 after placement of the skirt device 90, the cannula 100 can also be removed from the organ wall 102 in the same fashion without removing the skirt device 90.

It is further understood according to certain implementations that the skirt device 90 does not place any substantial forces on the organ tissue, including the organ wall 102. That is, the only forces of any note would be the compression fit of the skirt device 92 and stem 96 around the cannula 100 that results in the separation or isolation (including, in some embodiments, a substantially fluidic seal) as discussed above.

In use, a protection device (such as skirt device 20 or skirt device 40) according to any embodiment herein can be used in combination with a heart assist device to fluidically isolate the interface between the cannula inserted into the heart chamber (such as the left ventricle) and the incision or opening formed for cannula insertion therethrough.

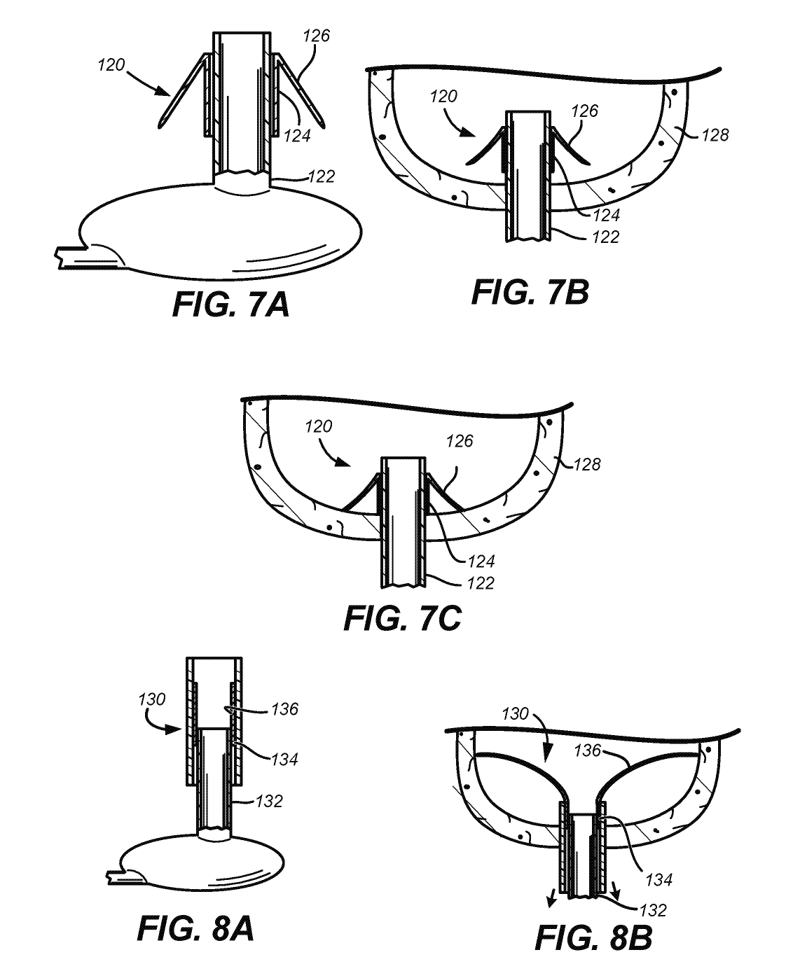

In those embodiments in which the protection device has a deployed configuration and an undeployed configuration as discussed above, the skirt device can initially be positioned in the undeployed configuration. According to certain implementations, the skirt device in the undeployed configuration is positioned around the cannula such that the cannula is disposed through the cannula opening and the outer edge of the skirt structure or sheet (according to any embodiment herein) is also disposed adjacent to, substantially adjacent to, near, or against the cannula (the retracted or undeployed configuration). For example, as depicted in FIG. 7A, the protection device 120 (which is similar—or identical—to the device 80 depicted in FIG. 5) is positioned over or around the cannula 122 such that the stem 124 is positioned against the outer surface of the cannula 122 and the flexible sheet 126 is coupled to a distal portion of the stem 124 and is disposed in an undeployed configuration in which the sheet 126 is disposed near or substantially adjacent to the stem 124 and the cannula 122 as shown.

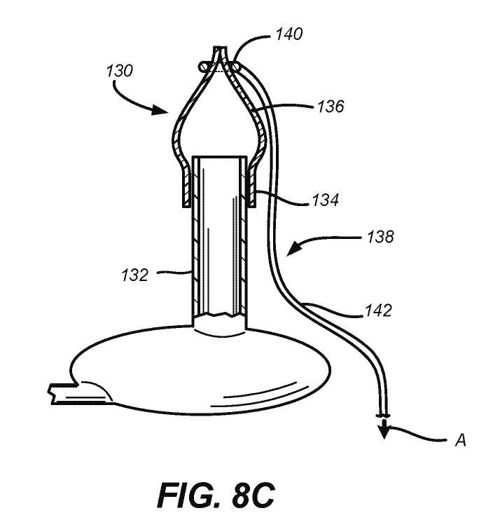

Alternatively, in other embodiments, such as that depicted in FIGS. 8A and 8C, the protection device 130 (which is similar—or identical—to the devices 60, 70 depicted in FIGS. 4A and 4B) in the undeployed configuration is positioned over or around or otherwise attached to the distal end of the cannula 132 such that the stem 134 is positioned against the outer surface of the cannula 132 and the flexible sheet 136 is coupled to a distal portion of the stem 134 such that the sheet 136 extends distally from the stem 134 past the distal end of the cannula 132 as shown in FIGS. 8A and 8C. More specifically, in FIG. 8A, the sheet 136 extends distally from the stem 134 such that the outer edge of the sheet 136 extends past the distal end of the cannula 132 while the sheet 136 and the stem 134 are substantially collinear as shown. Alternatively, in FIG. 8C, the sheet 136 extends distally from the stem 134 past the distal end of the cannula 132 and is cinched or otherwise held in a restrained (also referred to as “cinched”) position at a point distal of the distal end of the cannula 132 as shown until the sheet 136 is released from the restraint device 138 (as discussed in additional below).

In these embodiments, then the skirt device can be moved into the deployed or expanded configuration for use. For example, as depicted in FIG. 7C, the flexible sheet 126 of the protection device 120 is disposed in the deployed configuration in which the sheet 126 (that was disposed adjacent to the stem 124 in the undeployed configuration) extends radially away from the stem 124 and the cannula 122 such that the outer edge of the sheet 126 is proximal of the distal end of the cannula 132 as shown, thereby forming a substantially conical shape as shown. Alternatively, as shown in FIG. 8B, the flexible sheet 136 of the protection device 130 is disposed in the deployed configuration in which the sheet 136 (that was disposed extending distally of the distal end of the cannula 132) extends radially away from a point distal of the cannula 132 such that the outer edge of the sheet 136 is distal of the distal end of the cannula 132 as shown, thereby forming a substantially reverse conical shape as shown.

In the embodiment depicted in FIGS. 7A-7C, an additional step is provided, according to certain implementations. That is, upon insertion of the cannula 122 and the protection device 120 through the wall 128 of the organ (such as, for example, the heart), the cannula 122 is urged distally a sufficient distance into the organ as best shown in FIG. 7B such that the entire flexible sheet 126 is urged past the wall 128. As such, the flexible sheet 126 thereby “clears” the wall 128 such that the sheet 126 can extend radially into the deployed configuration as shown in FIG. 7B. Once the flexible sheet 126 is urged past the wall 128 such that it can move into the deployed configuration, the cannula 122 and protection device 120 can be retracted proximally until the protection device 120 is seated or otherwise positioned as desired with the outer edge of the flexible sheet 126 in contact with the inner surface of the wall 128 as shown in FIG. 7C such that the cannula 122/incision interface is isolated.

The restraint device 138 of FIG. 8C will now be discussed. The device 138 according to one implementation as shown is a suture cinch device 138 having a cinching loop 140 at the distal end that can be positioned or disposed around the flexible sheet 136 and a cinching length 142 of the device 138. The cinching length 142 can be urged proximally as represented by arrow A to cause the cinching loop 140 to tighten around the sheet 136 as shown. As such, the device 138 can be used to restrain the flexible sheet 136 in its undeployed configuration as shown in FIG. 8C while the protection device 130 is being positioned through the wall of the target organ (not shown). Once the device 130 is disposed as desired, the cinching loop 140 can be loosened by urging or allowing the cinching length 142 to move distally, thereby loosening the loops 140 and allowing the flexible sheet to move toward its deployed configuration. Alternatively, the restrain device 138 can be any known device that can be used to restrain the protection device 130 in its undeployed configuration.

In this deployed configuration (as shown in FIGS. 7B and 8B and discussed above), the cannula opening is still in contact with or positioned against the cannula but the outer edge of the sheet/skirt structure expands radially such that the skirt device takes on its conical or reverse conical shape as discussed above and depicted in the figures and makes it possible for the outer edge to contact the inner wall around and at some distance axially from the incision, thereby isolating the interface between the cannula and the incision as discussed in detail above.

In such embodiments (using a deployable skirt device), the skirt device can be attached to the cannula in its undeployed configuration prior to insertion of the cannula through the incision and into the organ chamber. Once the cannula is positioned as desired through the incision, the skirt device can be deployed and positioned or attached such that the isolation is created.

Alternatively, the protection device can be inserted through the incision first (before the cannula), and then the cannula can be inserted through the incision and the cannula opening of the skirt device at the same time. In such an embodiment, the skirt device can be deployed into its deployed configuration first (before the cannula is inserted) or it can be deployed after the cannula is positioned therethrough.

Alternatively, if the skirt device does not have both deployed and undeployed configurations, the skirt device can be positioned in the chamber prior to insertion of the cannula.

According to any embodiment disclosed or contemplated herein, any skirt device herein can have one or more radiopaque markers thereon or therein or be made of material that is radiopaque or has radiopaque additives. It is understood that the radiopaque feature in any embodiment allows for assisting with the positioning of the device during surgery and the assessment of positioning after implantation via radiography techniques (such as, for example, fluoroscopy).

In certain specific implementations, the sheet or skirt structure according to any embodiment disclosed or contemplated herein can have a textured surface on either side or both sides. The textured surface can, in certain embodiments, promote tissue ingrowth and reendothelialization. Further, the textured surface on the blood side of the skirt structure can prevent thrombus formation by inducing neointimal tissue on the device. In addition, the textured surface on the skirt side can promote rapid incorporation and mechanical connection to the heart wall and help promote rapid healing of and around the wound made by insertion of the cannula into the heart. The textured surface can be created by incorporation of Dacron/polyester or some other polymeric material onto the otherwise smooth sheet surface or by molding the device with rough textured surfaces incorporated therein. Alternatively, any other known texturing method can be used.

Further, with respect to any implementation disclosed or contemplated herein, an adhesive can be applied to the skirt structure/sheet along a portion of the structure that is in contact with the inner wall of the organ wall such that the skirt structure adheres to the inner organ wall. This can prevent movement of the skirt device in relation to the organ wall and assist in establishing more effective isolation of the wound where the cannula is positioned through the organ wall. It is understood that various known adhesives could be used, including BioGlue® (glutaraldehyde and bovine serum albumi), gelatin and thrombin mixtures, polyethylene glycol polymers, and the like.

In certain embodiments, the skirt device in accordance with any implementation disclosed or contemplated herein is a separate component that can be used in combination with a heart assist device as described above. Alternatively, the skirt device can be an integral part of the cannula such that it is already attached to the cannula and is inserted through the incision and into the organ chamber as described above.

As mentioned above, it is understood that the various protection device embodiments disclosed or contemplated herein can be used with other known circulatory support devices in addition to LVADs, such as RVADs and any other known pump or pumping mechanism/device that draws blood from any chamber of the heart, including any support device in which the pump is disposed within the cannula of the support device.

Although the present invention has been described with reference to preferred embodiments, persons skilled in the art will recognize that changes may be made in form and detail without departing from the spirit and scope of the invention.

What is claimed is:

1. A protection device for isolating a cannula/incision interface from organ chamber fluid, the device comprising:
(a) a flexible sheet; and
(b) an inner opening defined by the sheet, wherein the inner opening is sized to receive a cannula such that contact is established between the sheet and the cannula when the cannula is disposed through the inner opening,
wherein a portion of the sheet is contactable with an inner wall of an organ chamber.

2. The protection device of claim 1, wherein the contact between the sheet and the cannula is snug contact.

3. The protection device of claim 1, wherein the portion of the sheet is an outer edge of the sheet, wherein the outer edge is snuggably contactable with the inner surface of the organ chamber.

4. The protection device of claim 1, further comprising at least two ribs operably coupled to the flexible sheet.

5. The protection device of claim 4, wherein each of the at least two ribs extends from the inner opening to the outer edge of the flexible sheet.

6. The protection device of claim 5, wherein the at least two ribs are constructed and arranged to move between a retracted position in which the at least two ribs and the flexible sheet are disposed adjacent to the cannula and a deployed position in which the outer edge is in contact with the inner wall of the organ chamber.

7. The protection device of claim 1, further comprising a stem operably coupled to the flexible sheet, wherein the stem defines an opening sized to receive the cannula.

8. The protection device of claim 7. wherein the stem is operably coupled to the flexible sheet at or near the inner opening.

9. The protection device of claim 7, wherein the stem comprises an anchor disposed at or near a proximal end of the stem.

10. The protection device of claim 9, wherein the anchor comprises a lip.

11. A protection device for use with a cannula disposed through a wall of an organ, the device comprising:
(a) a flexible sheet comprising a retracted configuration and a deployed configuration;
(b) a cannula opening defined in the sheet, the cannula opening sized to receive the cannula; and
(c) a contactable portion of the circular sheet, the contactable portion being shaped to be contactable with an inner wall of the organ when the sheet is in the deployed configuration.

12. The protection device of claim 11, wherein the contactable portion snugly contacts the inner wall of the organ when the sheet is in the deployed configuration.

13. The protection device of claim 11, wherein the contactable portion forms a substantially fluidic seal with the inner wall of the organ when the sheet is in the deployed configuration.

14. The protection device of claim 11, wherein the deployed configuration comprises a substantially conical or rounded shape.

15. The protection device of claim 11, wherein the deployed configuration comprises a substantially flat shape.

16. The protection device of claim 11, wherein the cannula opening is sized to receive the cannula in a snug coupling.

17. A protection device for use with a cannula configured for insertion through a wall of an organ of a patient, the device comprising:
(a) a flexible, substantially circular sheet;
(b) a cannula opening defined in the sheet, the cannula opening sized to receive the cannula in a snug coupling;
(c) an outer edge of the circular sheet; and
(d) at least two ribs operably coupled to the circular sheet, wherein each of the at least two ribs extends from an edge of the cannula opening to the outer edge of the circular sheet, wherein the at least two ribs are constructed and arranged to move between a retracted position in which the at least two ribs and the circular sheet are disposed adjacent to the cannula and a deployed position in which the outer edge is in contact with an inner wall of the organ.

18. The protection device of claim 17, wherein the outer edge is in snuggable contact with the inner wall of the organ in the deployed position.

19. The protection device of claim 17, further comprising a stem operably coupled to the flexible sheet at or near the cannula opening, wherein the stem defines an opening sized to receive the cannula.

20. The protection device of claim 19, wherein the stem comprises an anchor disposed at or near a proximal end of the stem.
